(19) United States
(12) Patent Application Publication
(10) Pub. No.: US 2023/0324423 A1
(43) Pub. Date: Oct. 12, 2023

(54) DIAGNOSTIC LABORATORY SYSTEMS, ANALYZER INSTRUMENTS, AND CONTROL METHODS

(71) Applicant: Siemens Healthcare Diagnostics Inc., Tarrytown, NY (US)

(72) Inventors: Dariusz Tarnawski, Hackettstown, NJ (US); Gerard Killeen, Nenagh County Tipperary (IE); Elizabeth Kozak, Miami, FL (US)

(73) Assignee: Siemens Healthcare Diagnostics Inc., Tarrytown, NY (US)

(21) Appl. No.: 18/044,168

(22) PCT Filed: Oct. 6, 2021
(86) PCT No.: PCT/US2021/071743

§ 371 Date: Mar. 6, 2023

Related U.S. Application Data
(60) Provisional application No. 63/109,173, filed on Nov. 3, 2020.

(51) Int. Cl. G01N 35/00; G05B 15/02; G16H 10/40

(52) U.S. Cl. CPC G01N 35/00871; G05B 15/02; G16H 10/40; G01N 2035/00326

(57) ABSTRACT

Methods of controlling diagnostic laboratory systems include providing one or more modules, each of the one or more modules configured to process a specimen container and/or analyze a specimen; providing middleware configured to communicate with the one or more modules, wherein the middleware is configured to generate instructions to change an operational state of at least one of the one or more modules to enabled or disabled; generating, by the middleware, one or more instructions to change the operational state of at least one of the one or more modules; and changing the operational state of at least one of the one or more modules in response to one or more instructions generated by the middleware. Systems including a middleware server configured to carry out the methods are provided as are other aspects.

### FIELD

Embodiments of this disclosure relate to diagnostic laboratory systems, analyzer instruments, and methods that provide control of such systems and instruments.

### BACKGROUND

Centralization and consolidation of multiple small-scale diagnostic laboratories into larger-scale diagnostic laboratories for the analysis of bio-fluid specimens (e.g., blood, blood plasma, blood serum, urine, cerebrospinal fluid, etc.) has been a trend in recent years. In operation, large-scale diagnostic laboratories may process millions of bio-fluid specimens each year across a large number of laboratory analyzers (e.g., 20+). In addition to the laboratory analyzers, there may be ancillary test processing equipment (e.g., ancillary modules) such as one or more specimen container loader devices, specimen container unloader devices, or combined input/output (I/O) loader devices, desealers, centrifuges, quality check modules, decappers, aliquoters, and the like that preprocess the specimens and/or specimen containers before they arrive at a laboratory analyzer for testing of the specimens.

Many of the laboratory analyzers may have similar or overlapping capabilities in that they may run a large number of the same or differing test menus thereon. The operations of such large-scale diagnostic laboratories undergo continuous monitoring, evaluation, and intervention/manipulation by human operators. This may be time consuming and inaccurate.

### SUMMARY

According to a first aspect, a method of controlling a diagnostic laboratory system is provided. The method includes providing one or more modules, each of the one or more modules configured process a specimen container and/or analyze a specimen; providing middleware configured to communicate with the one or more modules, wherein the middleware is configured to generate instructions to change an operational state of at least one of the one or more modules to at least enabled or disabled; generating, by the middleware, one or more instructions to change the operational state of at least one of the one or more modules; and changing the operational state of at least one of the one or more modules in response to one or more instructions generated by the middleware.

In a further aspect, a method of controlling a diagnostic instrument is provided. The method includes providing a plurality of modules in the diagnostic instrument, the plurality of modules including a master module and one or more submodules, each of the plurality of modules configured to process a specimen container and/or analyze a specimen; generating first instructions to change an operational state of a first module of the plurality of modules; transmitting the first instructions to the master module; generating second instructions, by the master module, to change the operational state the first module in response to the first instructions; and changing the operational state of the first module in response to the second instructions.

In another aspect, a diagnostic instrument is provided. The diagnostic instrument includes a master module; and one or more submodules in communication with the master module, wherein the master module is configured to receive first instructions to change an operational status of at least one of the one or more submodules and to generate second instructions to change the operational status of at least one of the one or more submodules in response to the first instructions, and wherein the one or more submodules are configured to change operational state in response to the second instructions.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are for illustrative purposes and are not necessarily drawn to scale. Accordingly, the drawings and descriptions are to be regarded as illustrative in nature, and not as restrictive. The drawings are not intended to limit the scope of the disclosure in any way.

FIG. 1 illustrates a schematic block diagram of a diagnostic laboratory system including a plurality of laboratory analyzers, ancillary modules, and middleware according to one or more embodiments.

FIG. 2 illustrates a schematic block diagram of a diagnostic laboratory system including diagnostic instruments, laboratory analyzers, ancillary modules, and middleware according to one or more embodiments.

FIG. 3 illustrates a side elevation view of a specimen container containing a specimen and located within a carrier according to one or more embodiments.

FIG. 4 illustrates a diagnostic instrument of a diagnostic laboratory system including a master module and submodules according to one or more embodiments.

FIG. 5 illustrates a block diagram showing a portion of communications within a diagnostic laboratory system according to one or more embodiments.

FIG. 6 illustrates components within and coupled to a master module of a laboratory instrument according to one or more embodiments.

FIG. 7 is a flowchart illustrating a method of controlling a diagnostic laboratory system according to one or more embodiments.

FIG. 8 is a flowchart illustrating a method of controlling a diagnostic instrument of a diagnostic analyzer system according to one or more embodiments.

### DETAILED DESCRIPTION

Diagnostic laboratory systems include a plurality of modules configured to process specimen containers and/or analyze specimens (e.g., blood and other body fluids) located within the specimen containers. Examples of modules (e.g., ancillary modules) configured to processes specimen containers include input/output (I/O) loaders, desealers, and decappers. Examples of ancillary modules configured to process specimens prior to testing include centrifuges, quality control modules, and aliquoters. Examples of modules configured to analyze specimens include laboratory analyzers (sometime referred to herein simply as “analyzers”) that analyze components within the specimens or test for specific chemicals. Some modules may be configured to perform functions of ancillary modules and analyzers. In some embodiments, some modules may be configured to perform functions of only ancillary modules and other modules may be configured to perform functions of only analyzers.

The diagnostic laboratory systems may include a track or the like that transports specimen containers between the different modules. In some embodiments, the modules may include tracks such that the modules may be coupled together or be proximate one another to form a continuous track between some or all the modules. Thus, a diagnostic laboratory system may include a plurality of modules physically coupled together with the track extending between (e.g. through) the modules. In other embodiments, the laboratory analyzer systems may include a track with modules coupled to the track or located proximate the track. In such embodiments, the modules may be spaced from one another. The specimen containers may be diverted from the track or removed from the track to enter or access one or more of the modules. Some modules may be remote from the track.

Some diagnostic laboratory systems include diagnostic instruments that may or may not be attached to a track. An instrument includes a plurality of modules that are mechanically interconnected and may be controlled at least in part by a processor. In some embodiments, an instrument may include a processor or the like that controls and receives status of the modules of the instrument. In some embodiments, the modules of an instrument are mechanically interconnected by a track that may be coupled to the track in a diagnostic laboratory system. In other embodiments, the instrument may include a plurality of modules that are connected together and remove specimen containers from a track and perform testing or processes on the specimens and/or the specimen containers. Thus, an instrument may include one or more analyzers and/or one or more ancillary modules.

Each of the analyzers may perform one or more tests (e.g., assays) on the specimens. For example, some analyzers may perform one or more clinical chemical analysis, other analyzers may perform one or more immunoassays, and other analyzers may perform one or more other functions, such as genetic analysis or drug analysis. In some embodiments, a single analyzer may perform a plurality of different tests. For example, in some embodiments, a first analyzer may perform a menu of tests A-C and a second analyzer may perform a menu of tests D-F. A third analyzer may perform a menu of tests G-H. In some embodiments, test menus of different analyzers may overlap or be the same. For example, a fourth analyzer may also perform a menu of tests A-C identical to first analyzer. Thus, in some embodiments, a plurality of analyzers and/or ancillary modules may perform the same tests or processes for redundancy purposes.

A medical professional may order certain tests (e.g., test orders) to be performed on certain specimens (e.g., fluids) from a patient. These test orders may be entered into a program or server, such as a hospital information system (HIS). For example, the medical professional may order a plurality of tests to be performed on blood or serum of a patient. The test orders may be transmitted from the HIS to a laboratory information system (LIS) that receives a plurality of test orders and designs testing protocols and/or scheduling for a diagnostic laboratory system to complete the test orders. The test orders may come from sources other than a HIS. Middleware software (e.g., middleware) may run on a middleware server or other computer or server that is in electronic communication with the diagnostic laboratory system. The middleware instructs the modules to run the testing protocols designed by the LIS and may perform other functions as described herein. For example, the middleware may include one or more software programs that assist with inventory and load control of the analyzers and/or the ancillary modules.

In some embodiments, the middleware may monitor the modules and report their status to the LIS. The middleware may also output the status and other operating information to a user of the diagnostic laboratory analyzer. For example, if a module is experiencing problems, the module may output information regarding the problem to the middleware, which may then output the information to the LIS. The LIS may redesign testing protocols and/or testing schedules in response to the status information of the module.

The laboratory diagnostic systems described herein include middleware or other programs that generate instructions that change the operational states of one or more of the modules. The operational states of the modules may include, enabled, disabled, and a sleep state. In some embodiments, the middleware generates instructions that cause one or more of the modules to be disabled or enabled. For example, if a problem is detected with a specific module, the middleware can generate instructions that disable that specific module and, in some embodiments, reroute specimen containers to other modules for testing and/or processing provided there is sufficient redundancy in the system. In some embodiments, the middleware can disable a module if the module is not going to be used. For example, during periods where no tests are scheduled to be run on a specific module, that specific module can be disabled, which reduces wear and tear on the module and reduces energy consumption.

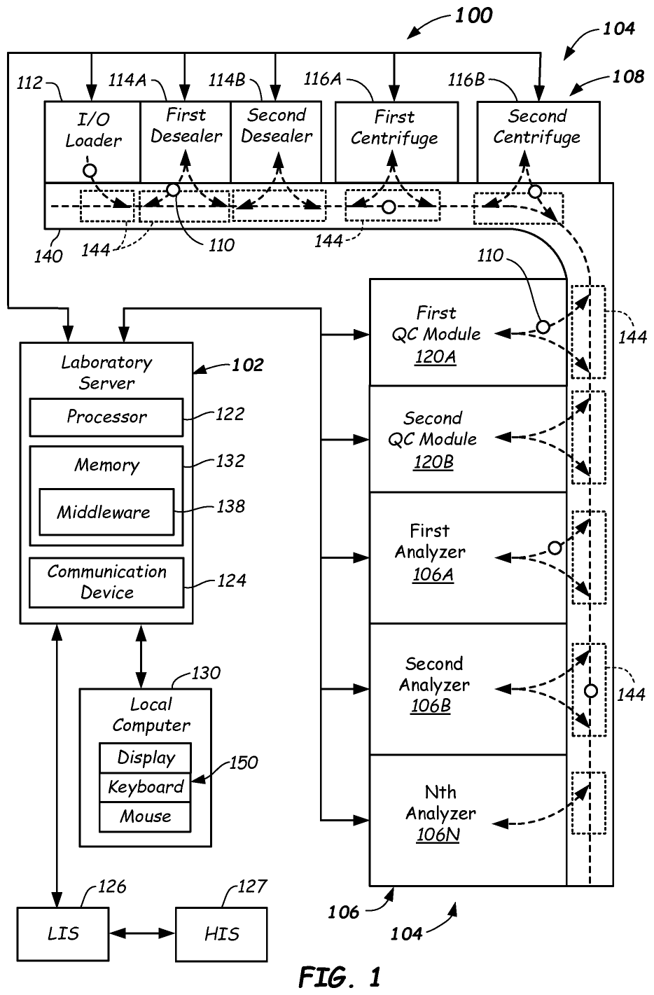

Some embodiments of diagnostic laboratory systems may include one or more instruments. One module in an instrument may be a master module. The master module includes circuitry, software, or other devices that enable to the master module to change operational states of the other modules (e.g., submodules) of the instrument. In some embodiments, the master module may physically switch the operational states of the other modules, such as to enabled, disabled, or sleep. In other embodiments, the master module may transmit instructions to the other modules that cause the other modules to change operational states. The master module may be in communication with a server or software, such as middleware, that instructs the master module to change the operational state of specific submodules in the instrument. These and other methods and systems are described herein with reference to FIGS. 1-8 herein.

Reference is now made to FIG. 1, which illustrates a diagnostic laboratory system 100 according to embodiments of the disclosure. The diagnostic laboratory system 100 may automatically process large numbers of specimens (e.g., biological samples) with minimal human intervention, except possibly for the introduction of STAT tests and maintenance and service for breakdowns and work stoppages. The diagnostic laboratory system 100 may include a laboratory server 102 communicatively coupled to a plurality of modules 104. The laboratory server 102 may control the operation and/or scheduling of some or all aspects of one or more of the modules 104. The laboratory server 102 may be a computer, for example, and may be located proximate or remote from the modules 104. One or more of the modules 104 may include a local workstation or controller configured to control operation thereof for carrying out various types of testing and/or processing on specimens and/or specimen containers thereon.

The modules 104 may comprise a plurality of laboratory analyzers 106 (referred to herein as “analyzers” and represented by a first analyzer 106A, a second analyzer 106B, and an Nth analyzer 106N) and a plurality of ancillary modules 108 (e.g., ancillary test processing equipment). The diagnostic laboratory system 100 may include any number of analyzers 106 and ancillary modules 108. In the embodiment of FIG. 1, there are N analyzers, three of which are illustrated as a first analyzer 106A, a second analyzer 106B, and an Nth analyzer 106N. The diagnostic testing carried out on the analyzers 106 can include, but is not limited to, immunoassay testing (e.g., chemiluminescent immunoassays (CLIA), radioimmunoassays (RIA), counting immunoassays (CIA), fluoroimmunoassays (FIA), and enzyme immunoassays (EIA and including enzyme linked immunosorbent assays (ELISA)) to target specific target biomolecules. In addition, some of the analyzers 106 may measure concentrations of substances (e.g., glucose, hemoglobin A1C, lipids (fats), triglycerides, blood gases (e.g., carbon dioxide, etc.), enzymes, electrolytes (e.g., sodium, potassium, chloride, and bicarbonate), lipase, bilirubin, creatinine, blood urea nitrogen (BUN), hormones (e.g., thyroid stimulating hormone), hepatitis, minerals (e.g., iron. calcium, magnesium, etc.), proteins, and other metabolic products and the like in the specimens. Other testing may be performed on the specimens by the analyzers 106. The specimens can include whole blood, serum, plasma, urine, cerebral-spinal fluid, interstitial fluid, saliva, feces, and the like.

In some embodiments, two or more of the analyzers 106 may be capable of performing the same tests (i.e., they have the same or overlapping test menus), while others of the analyzers 106 may be capable of performing only a limited number of tests or only certain individual tests. For example, the first analyzer 106A and the second analyzer 106B may be configured to run tests A-D and the Nth analyzer 106N may be configured to run tests E-G. In another example, the first analyzer 106A may be configured to run tests A-E, the second analyzer 106B may be configured to run tests D-F, and the Nth analyzer 106N may be configured to run test F. Thus, in some embodiments, the analyzers 106 may be configured to run the same or overlapping tests, which enable the diagnostic laboratory system 100 to handle high test volumes, perform redundant testing, and continue testing in the event an analyzer becomes nonfunctional or disabled.

The ancillary modules 108 may include various modules or machines that are configured to prepare and/or process specimen containers 110 (a few labelled) and/or specimens located therein for testing. In some embodiments, the ancillary modules 108 prepare the specimen containers 110 and/or the specimens to be received and/or tested by the analyzers 106. In the embodiment of FIG. 1, the ancillary modules 108 include an input/output (I/O) loader 112, a first desealer 114A, a second desealer 114B, a first centrifuge 116A, a second centrifuge 116B, a first quality check (QC) station 120A (e.g., a first QC module), and a second QC station 120B (e.g., a second QC module). The diagnostic laboratory system 100 may include other or fewer ancillary modules. In the embodiment of FIG. 1, the diagnostic laboratory system 100 may have redundant ancillary modules to handle high test volumes and to enable testing in the event an ancillary module becomes nonfunctional or disabled.

The modules 104 may be electrically coupled to the laboratory server 102. For example, the modules 104 may be in electronic communication with the laboratory server 102 by wired and/or wireless networks such as by WAN, LAN, or WIFI. The laboratory server 102 may be implemented in a computer, for example, that may or may not be proximate the modules 104. In some embodiments, the laboratory server 102 may be remote from other components of the diagnostic laboratory system 100, such as in a different building or even in a different state. The laboratory server 102 may include a processor 122 that controls components of the laboratory server 102 and/or the modules 104 as described herein. For example, the processor 122 may execute computer programs stored in a memory 132 that control components of the laboratory server 102 and/or control or schedule activities (e.g., tests, pre-screening, or pre-processing) on the modules 104.

The laboratory server 102 may include a communication device 124 that enables communications between the laboratory server 102 and the modules 104. The communication device 124 may provide wireless communications (e.g., radio frequency (RF) or optical communications) and/or wired communications with components of the diagnostic laboratory system 100. The laboratory server 102 may be in communication with a laboratory information system (LIS) 126 and a local computer 130 as described in greater detail below. The local computer 130 may include a user interface 150, which may include a display, a keyboard, and a mouse. The user interface 150 enables a user to input data into the diagnostic laboratory system 100 and to receive information regarding the status of the diagnostic laboratory system 100 or components thereof. In some embodiments, the local computer 130 may be a portable or wireless device and the user interface may be or include a touchscreen.

The laboratory server 102 may include memory 132 that stores computer programs in the form of computer code that is executable by the processor 122. One of the computer programs may be middleware 138 that monitors the status of the modules 104 and movement of the specimen containers 110 between the modules 104. In some embodiments, the middleware 138 may be executed on a dedicated server or computer, such as a middleware server. The middleware 138 may also control scheduling of workloads of the diagnostic laboratory system 100 that may be designed by the LIS 126. For example, the middleware 138 may control the timing of tests on the specimens to accomplish one or more objectives, such as providing a balanced workload among the modules 104.

In some embodiments, the LIS 126 may be in communication with a hospital information system (HIS) 127. The HIS 127 may be one or more programs and/or servers that enable medical professionals or the like to enter test orders. The test orders describe the tests that are to be performed on specific specimens. When the specimens are received by the diagnostic laboratory system 100, the LIS 126 may receive the test orders from the HIS 127 and design testing protocols for the diagnostic laboratory system 100.

The middleware 138 may receive input regarding the status of the modules 104 to control or balance the workload among the modules 104. In some embodiments, the middleware 138 may receive a status indicating at least one parameter of one or more of the modules 104. An operational state of at least one of the modules 104 may be changed by or set by the middleware 138. As described herein, the middleware 138 may receive test results of a quality control test performed by one or more of the ancillary modules 108 or one or more of the analyzers 106. The middleware 138 may determine whether a test result is acceptable to forward to the LIS 126.

The middleware 138 may also control the operational state of the modules 104, such as enabling and disabling specific modules 104 as described herein. Enabling a module 104 includes enabling the module 104 to receive specimens and/or specimen containers 110 and preform tests or processes thereon, which the module is intended to perform. Disabling a module may include preventing the disabled module from receiving specimens and/or specimen containers 110. Disabling a module may also prevent the disabled module from performing tests or processes, which the module is intended to perform. For example, the middleware 138 may generate instructions (e.g., first instructions) to disable the second analyzer 106B and cause the specimen containers 110 to be routed away from the second analyzer 106B. In a like process, the middleware 138 may generate instructions to enable the second analyzer 106B and cause the specimen containers 110 to be diverted to the second analyzer 106B. The middleware 138 may generate instructions to enable and disable other modules.

A track 140 may be configured to transport the specimen containers 110 between the modules 104. The track 140 may be a railed track (e.g., a mono rail or a multiple rail), a collection of conveyor belts, conveyor chains, moveable platforms, magnetic transportation, or any other suitable type of conveyance mechanism. The track 140 may be circular or other suitable shapes and may be a closed track (e.g., an endless track), and may have paths as offshoots from a main track in some embodiments. In some embodiments, the modules 104 and the track 140 may be configured so as to accommodate the diagnostic laboratory system 100 within a laboratory. In some embodiments, the diagnostic laboratory system 100 may be very large. For example, the track 140 may be 100 m or longer in length.

One or more gate mechanisms 144 (e.g., flow diverters—a few labelled) may be located on, proximate, or incorporated within the track 140 proximate one or more of the modules 104. The gate mechanisms 144 divert the specimen containers 110 into and out of enabled modules and prevent specimen containers 110 from entering disabled modules.

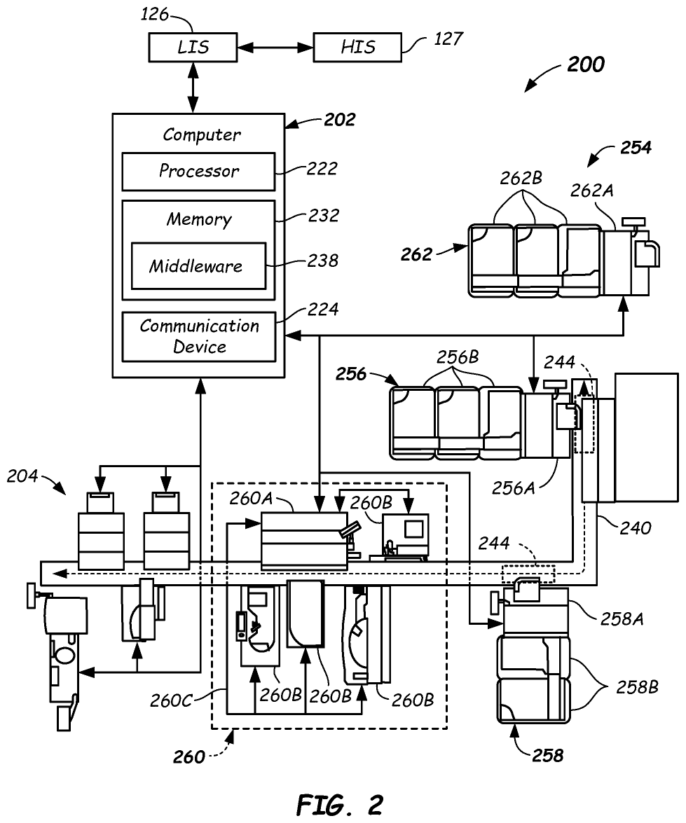

Additional reference is now made to FIG. 2, which illustrates a diagnostic laboratory system 200 that includes instruments 254 (e.g., diagnostic instruments). In the embodiment of FIG. 2, the diagnostic laboratory system 200 includes four instruments as described herein. In other embodiments, the diagnostic laboratory system 200 may include one or more instruments. In some embodiments, one or more of the instruments may include a plurality of modules that function similar to the modules 104 (FIG. 1).

The diagnostic laboratory system 200 may include a first instrument 256 (e.g., a first diagnostic instrument) and a second instrument 258 (e.g., a second diagnostic instrument) that each include two or more modules that are physically coupled together. The first instrument 256 and the second instrument 258 are shown being adjacent a track 240. In the embodiment of FIG. 2, the first instrument may include a master module 256A and one or more submodules 256B. In the embodiment of FIG. 2, the first instrument 256 includes three submodules 256B. In the embodiment of FIG. 2, the second instrument 258 includes a master module 258A and two submodules 258B.

The diagnostic laboratory system 200 may also include a third instrument 260 that includes a master module 260A and one or more submodules 260B that are not physically coupled to each other, but that may be proximate the track 240. The submodules 260B may include analyzers and/or ancillary modules. The master module 260A is configured to communicate with the submodules 260B via communication 260C, which may be wired or wireless communications.

The diagnostic laboratory system 200 may also include a fourth instrument 262 that may be remote from the other instruments. For example, the fourth instrument may be remote from the track 240 or located in a different building or facility than the other instruments. In some embodiments, the fourth instrument 262 may be self-contained wherein the fourth instrument 262 receives specimens and/or specimen containers by means other than a track. In some embodiments, the fourth instrument 262 may be coupled to a track (not shown) that is independent of the track 240. The fourth instrument 262 includes a master module 262A and one or more submodules 262B. In the embodiment of FIG. 2, the fourth instrument 262 includes three submodules 262B.

The diagnostic laboratory system 200 may also include individual modules 204 that may be identical or similar to the modules 104 (FIG. 1). The individual modules 204 may be in communication with the computer 202. Instruction generated by the middleware 238 may change the operational status of one or more of the individual modules 204. The individual modules 204 may include similar modules and/or may perform similar functions as the modules 104. For example, the individual modules 204 may include an I/O loader that may be identical or similar to the I/O loader 112 and may receive and/or provide for movement of the specimen containers 110 into and/or out of the diagnostic laboratory system 200.

The master modules 256A, 258A, 260A, 262A of the instruments 254 are in communication with the computer 202 or the like that generates first instructions to change the operational states of specific modules in the instruments 254. The master module of the instrument containing a module (e.g., a first module) that is to have a change in operational state generates and transmits second instructions to the first module. The first module changes operational state in response to the second instructions. In the embodiment of FIG. 2, the master modules 256A, 258A, 260A, 262A are in communication with the computer 202 that generates the first instructions. Other devices (not shown) may generate the first instructions described herein.

In some embodiments, the computer 202 is similar to or identical to the laboratory server 102 (FIG. 1). The computer 202 may include a processor 222 that executes programs stored in a memory 232. In the embodiment of FIG. 2, the memory stores middleware 238. The middleware 238 may be similar or identical to the middleware 138 (FIG. 1). The middleware 238 and/or other programs executed by the computer 202 may generate the first instructions described herein. The computer 202 may include a communication device 224 that communicates with the master modules 256A, 258A, 260A, 262A. In some embodiments, the communication device 224 may communicate with the master modules 256A, 258A, 260A, 262A via wired and/or wireless communications.

Referring to both FIG. 1 and FIG. 2, the first instructions generated by the middleware 138, 238 may include data packets or the like. The first instructions may include data indicating which modules are to have their operational states changed. For example, if the first instructions indicate that the operational state of the first analyzer 106A is to be changed, the data of the instructions will indicate such. If the first instructions indicate that an operational state of one of the submodules 256B in the first instrument 256 is to be changed, the data of the first instructions will indicate such and will be sent to the master module 256A. The master module 256A will generate second instructions to change the operational state of the submodule in response to the first instructions.

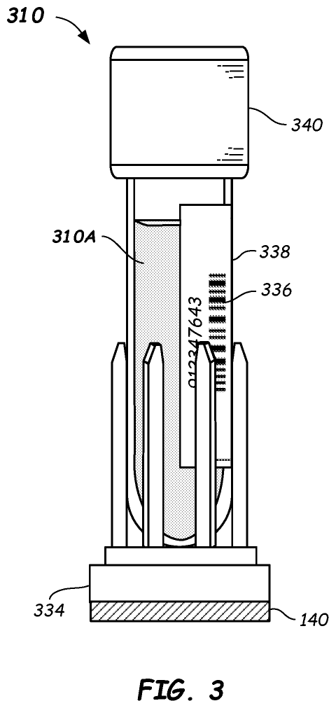

Additional reference is made to FIG. 3, which illustrates a side elevation view of a specimen container 310 containing a specimen 310A and located within a carrier 334. The specimen container 310 is an example of the specimen containers 110 (FIGS. 1-2). The carrier 334 transports the specimen container 310 throughout the diagnostic laboratory system 100, 200 as described herein. In some embodiments, the I/O loader 112 may read identifications, such as a bar code 336 located on the specimen container 310 and prepare the specimen container 310 for movement within the diagnostic laboratory system 100, 200. For example, the specimen container 310 may be provided with a label 338 that may include identification information thereon, such as, a time and/or date stamp, requested test(s), patient identification, the bar code 336, and the like. The identification information may be machine readable at various locations within the diagnostic laboratory system 100, 200. The specimen container 310 may include a cap 340 and may be sealed in some instances.

The first desealer 114A and the second desealer 114B and similar individual modules 204 or modules in the instruments 254 may deseal the specimen containers 110. The use of two desealers enables the diagnostic laboratory system 100, 200 to have redundant specimen pre-processing and handle large specimen volumes. The first centrifuge 116A and the second centrifuge 116B spin the specimen containers 110 to separate portions of the specimen 310A (fractionation). The diagnostic laboratory system 200 may include one or more centrifuges in the individual modules 204 and/or one or more of the instruments 254. In embodiments where the specimen is blood, the first centrifuge 116A and the second centrifuge 116B separate the blood and form a serum or plasma portion. The use of two centrifuges enables the diagnostic laboratory system 100, 200 to have redundant specimen pre-processing and handle large specimen volumes. The first QC module 120A and the second QC module 120B check the specimens for one or more characteristics, such as the presence of an interferent such as hemolysis, icterus, or lipemia (HIL) in the serum or plasma, or the presence of another interferent such as a blood clot, bubble, or foam therein. The diagnostic laboratory system 200 may include one or more QC modules in the individual modules 204 and/or one or more of the instruments 254. The use of two QC modules enables the diagnostic laboratory system 100, 200 to have redundant QC testing and handle large specimen volumes.

The track 240 may be configured to transport the specimen containers 110 between the different instruments 254 and different ones of the individual modules 204. The track 240 may be similar or identical to the track 140 (FIG. 1). The track 240 may be circular or other suitable shapes and may be a closed track (e.g., an endless track), and may have paths as offshoots from a main track in some embodiments. In some embodiments, instruments 254, the individual modules 204, and the track 240 may be configured to accommodate the diagnostic laboratory system 200 within a laboratory.

One or more gate mechanisms 244 (e.g., flow diverters —a few labelled) may be located on, proximate, or incorporated within the track 240 proximate one or more of the individual modules 204 and/or proximate one or more of the instruments 254. The gate mechanisms 244 may be similar or identical to the gate mechanisms 144 (FIG. 1) and may divert the specimen containers 110 into and out of enabled modules and certain ones of the instruments 254.

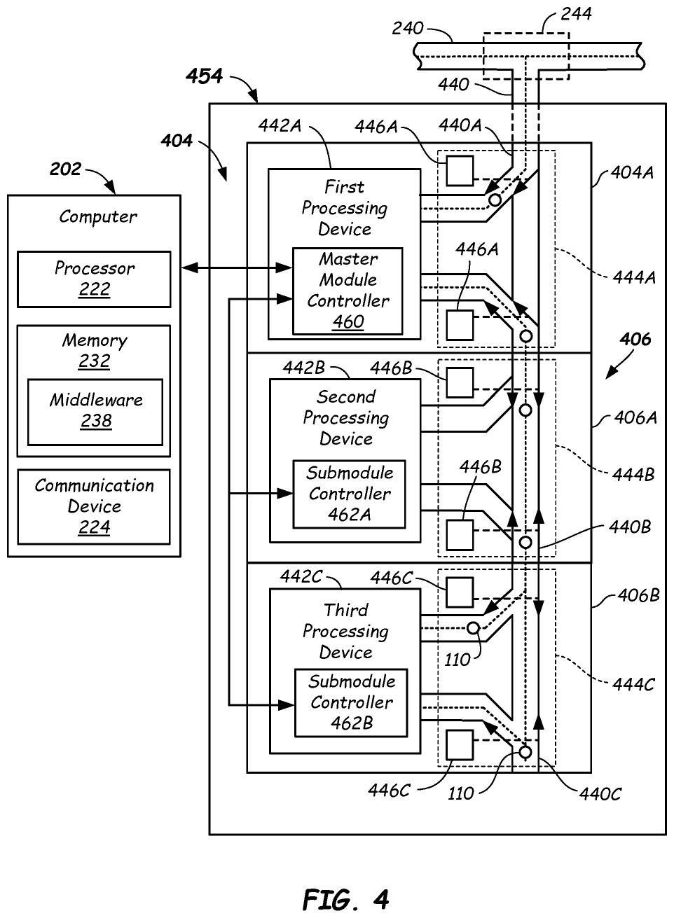

Additional reference is made to FIG. 4, which illustrates an instrument 454, which may be identical to or similar to one or more of the instruments 254 (FIG. 2). The instrument 454 includes two or more modules 404 including a master module 404A and one or more submodules 406. In the embodiment of FIG. 4, the instrument 454 includes two submodules that are referred to individually as a first submodule 406A and a second submodule 406B. In other embodiments, the instrument 454 may have a single submodule or more than two submodules. Each of the modules 404 may perform processes or analysis on specimen containers 110 and/or specimens therein. In some embodiments, some of the modules 404 may perform identical processes and/or analysis and in other embodiments, all the modules 404 may perform different analysis and/or processes. In some embodiments, the modules 404 may perform functions identical or similar to the modules 104 (FIG. 1) and/or the individual modules 204 (FIG. 2).

The instrument may include a track 440 that is coupled to the track 240. In some embodiments, a gate mechanism 244 may enable specimen containers 110 to be diverted to the instrument 454 when modules 404 within the instrument 454 are enabled and programed to process and/or analyze certain specimen containers or the specimens located therein. In a similar manner, the gate mechanism 244 may divert specimen containers 110 from entering the instrument 454 when no modules that would otherwise process and/or analyze certain specimen containers or specimens located therein are enabled. One or more of the modules 404 may have integral portions of the track 440 that pass through the modules 404.

In the embodiment of FIG. 4, the master module 404A includes a first processing device 442A, the first submodule 406A includes a second processing device 442B, and the second submodule 406B includes a third processing device 442C. The processing devices 442A-442C may be, for example, chemical testing devices and the like that process specimens and/or processing devices (e.g., ancillary devices) that prepare the specimens and/or the specimen containers 110 for processing or testing. In some embodiments, the master module 404A and the first processing device 442A receive specimen containers into the instrument 454. For example, the master module may read bar codes (e.g., bar code 336—FIG. 3) on the specimen containers 110. The master module 404A may perform other functions to prepare the specimen containers 110 and/or the specimens for analysis and/or processing.

In the embodiment of FIG. 4, a track first portion 440A is integral with or passes through the master module 404A, a second track portion 440B is integral with or passes through the first submodule 406A, and a third track portion 440C is integral with or passes through the second submodule 406B. In some embodiments, portions of the track 440 may pass proximate the one or more of the modules 404. A first gate mechanism 444A may be in or proximate the master module 404A, a second gate mechanism 444B may be in or proximate the first submodule 406A, and a third gate mechanism 444C may be in or proximate the second submodule 404C. The first gate mechanism 444A diverts the specimen containers 110 from or into the first processing device 442A, the second gate mechanism 444B diverts the specimen containers 110 from or into the second processing device 442B, and the third gate mechanism 444C diverts the specimen containers 110 from or into the third processing device 442C.

The first gate mechanism 444A is coupled to a first gate controller 446A that controls the first gate mechanism 444A, the second gate mechanism 444B is coupled to a second gate controller 446B that controls the second gate mechanism 444B, and the third gate mechanism 444C is coupled to a third gate controller 446C that controls the third gate mechanism 444C. In the embodiment of FIG. 4, the master module 404A is enabled, the first submodule 406A is disabled, and the second submodule 406B is enabled. Because the master module 404A is enabled, the first gate mechanism 444A is in an enabled state that diverts the specimen containers 110 into the first processing device 442A. Because the first submodule 406A is disabled, the second gate mechanism 444B is in a disabled state such that the specimen containers 110 are diverted from entering the second processing device 442B. Because the second submodule 406B is enabled, the third gate mechanism 444C is in an enabled state that diverts the specimen containers 110 into the third processing device 442C.

The master module 404A may include a master module controller 460, the first submodule 406A may include a submodule controller 460A, and the second submodule 406B may include a submodule controller 460B. The master module controller 460 may be in communication with the middleware 238. In some embodiments, the master module controller 460 may be in communication with the middleware 238 via the communication device 224 in the computer 202, for example. The master module controller 460 is in communication with the submodule controller 462A and the submodule controller 462B.

The master module controller 460 receives first instructions indicating which modules are to be enabled and disabled. In the embodiment of FIG. 4, the master module controller 460 receives the first instructions from the middleware 238. Other software or devices may generate and/or transmit the first instructions to the master module controller 460. The first instructions may be transmitted via conventional data protocols, such as data including packets. The master module controller 460 interprets the first instructions to determine the operational states of the modules 404. In some embodiments, the first instructions may provide information as to which operational state each of the modules 404 is to be in. In other embodiments, the first instructions may provide information indicating which modules 404 are to have changed operational states.

The master module controller 460 may generate second instructions that cause one or more of the modules 404 to change operational state to conform to the first instructions. The second instructions may be transmitted to the submodule controller 462A and/or the submodule controller 462B to change the operational state of the first submodule 406A and/or the second submodule 406B, respectively. In some embodiments, the master module controller 460, the submodule controller 462A, and the submodule controller 462B may be processors including or coupled to memory. The memory may store instructions that when executed by the processor, cause the respective module to change operational state. In other embodiments, the second instructions may be control signals that turn modules on (e.g., enable modules) and/or signals that turn modules off (e.g., disable modules). In some embodiments, the master module controller 460 may generate second instructions to change the operational status of the master module 404A.

In some embodiments, operational status of one or more of the modules 404 may be transmitted to the master module control 460, which may transmit the operational status from the instrument 454. For example, results from quality control tests performed by one or more of the modules 404 may be transmitted to the master module controller 460, which may then transmit the operational status from the instrument 454. In some embodiments, the operational status may be transmitted to the middleware 238. In such embodiments, the middleware 238 may enable and/or disable certain modules and/or certain instruments in response to the received operational status.

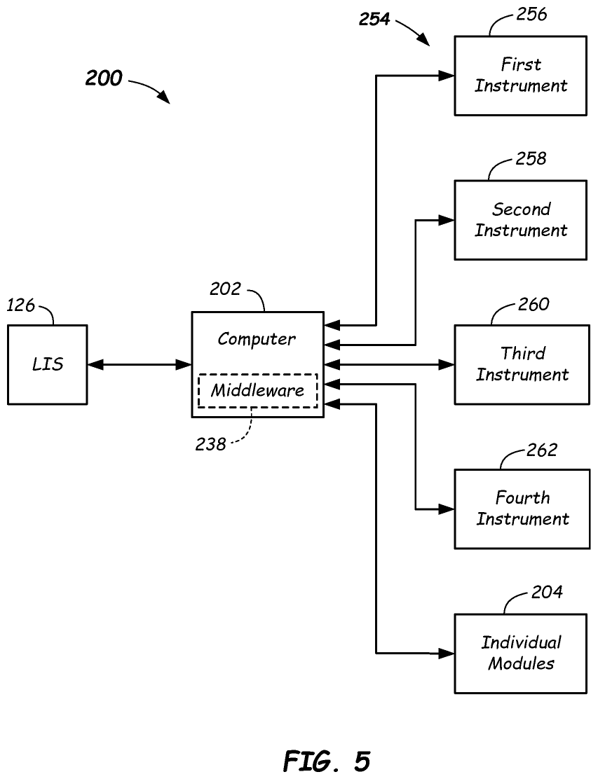

Additional reference is made to FIG. 5, which illustrates a block diagram showing a portion of communications within the diagnostic laboratory system 200. As shown in FIG. 5, the computer 202 may be electrically coupled to the first instrument 256, the second instrument 258, the third instrument 260, the fourth instrument 262, and one or more of the modules 204. Thus, the computer 202 is configured to communicate with one or more of the modules 204 and/or one or more of the instruments 254 of the diagnostic laboratory system 200. The middleware 238 located within the computer 202 may generate the first instructions to be transmitted to individual ones of the modules 204 and/or the instruments 254.

The computer 202 may transmit the first instructions to one or more of the instruments 254 and/or the individual modules 204 to change operational states of modules as described herein. As described herein, the first instructions may cause one or more of the instruments 254 and/or the individual modules 204 to change their operational state from enabled to disabled or from disabled to enabled. In some embodiments, the first instructions may instruct one or more of the instruments 254 and/or one or more of the individual modules 204 to enter a sleep state wherein the modules are disabled, but waiting for instructions to change their operational state to enabled. As described herein, the middleware 238 may generate the first instructions. In other embodiments, a user may provide an input to the middleware 238, such as via a local computer that causes the middleware 238 to generate the first instructions. The communication within the diagnostic laboratory system 200 may also enable the middleware 238 to monitor the operational state of one or more of the individual modules 204 and/or one or more of the instruments 254.

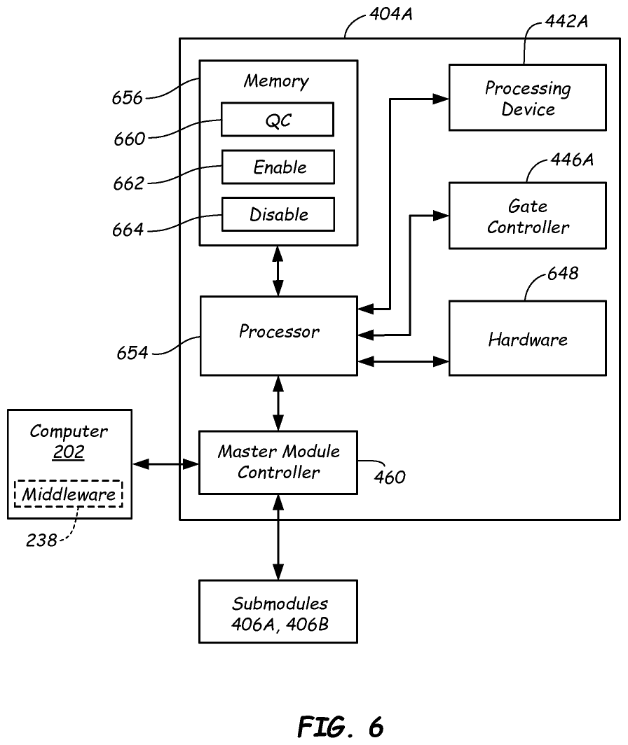

Additional reference is made to FIG. 6, which illustrates a block diagram of an embodiment showing components that may be within and coupled to the master module 404A. The master module 404A may include a processor 654 that is configured at least to execute one or more programs stored in memory 656. The processor 654 may be electrically coupled to the processing device 442A, the gate controller 446A, and other hardware 648.

In the embodiment of FIG. 6, the master module controller 460 is located separate from the processing device 442A. As shown in FIG. 6, the master module controller 460 may receive the first instructions from an external source, such as the computer 202. In the embodiment of FIG. 6, the master module controller 460 may communicate directly with the computer 202 and/or the middleware 238 running therein. The master module controller 460 may be in communication with the submodules and may generate the second instructions to set and/or change the operational state of one or more of the submodules.

The memory 656 may store one or more programs that may be executed by the processor 654. The programs may include a quality control (QC) program 660, an enable program 662, and a disable program 664. The QC program 660 may cause the master module 404A to run one or more QC routines to check the status of the master module 404A. For example, the QC routines may determine whether testing and/or analysis of specimens is accurate. In some embodiments, the QC routines may determine whether the master module 404A is able to process specimen containers. In some embodiments, the QC routines are self-testing routines.

The enable program 662 may enable or “turn on” the master module 404A from a disabled operational state in response to receiving the second instructions generated by the master module controller 460. In some embodiments, the master module 404A may be in a sleep operational state or the like wherein a minimum number of components are active when the master module 404A is in the sleep operational state. In the sleep operational state, the processor 654 and/or the master module controller 460 may be active to receive the first instructions to enable the master module 404A. In response to the enable instruction being received, the master module controller 460 may generate second instructions that cause the processor 654 to execute the enable program 662 to enable or turn on the master module 404A. In some embodiments, enabling the master module 404A may include activating the processing device 442A to perform analysis on specimens. In some embodiments wherein the master module 404A is an ancillary module, activating the processing device 442A may include preparing the processing device 442A to process specimen containers or specimens located therein for analysis.

In addition to enabling the processing device 442A, the enable program 662 may also enable the gate controller 446A to divert specimen containers to the processing device 442A. For example, when the master module 404A is in the disabled operating state, the gate controller 446A may prevent specimen containers from entering the master module 404A and/or accessing the processing device 442A. In some embodiments, the instruments 254 may operate faster when specimen containers 110 are diverted from or bypass disabled modules. In some embodiments, the enable program 662 may enable the hardware 648 to prepare the master module 404A to operate. For example, lights, sensors, fans, and other devices may be activated to enable the master module 404A to operate correctly in the enabled operational state.

The disable program 664 changes the operational state of the master module 404A from enabled to disabled. For example, the master module controller 460 receives first instructions that instruct the master module 404A to change the operational state to the disabled operational state. In response, the master module controller 460 may generate second instructions that cause the processor 654 to execute the disable program 664. The disable program 664 may cause the master module 404A to perform certain routines unique to the type of master module 404A in order to disable the master module 404A. For example, disabling routines for the ancillary modules may be different than for modules that are analyzers. Furthermore, the disabling routines may be different for different types of ancillary modules and different types of analyzers.

Execution of the disable program 664 may cause the first processing device 442A to shut down or enter a sleep state as described above. In some embodiments, the first processing device 442A may finish processing and/or analyzing specimens and/or specimen containers before shutting down. The specimens and/or specimen containers may then be removed from the first processing device 442A. For example, normal processing may proceed and the specimen containers 110 (FIG. 4) may be returned to the track 440 (FIG. 4) for further processing. In some embodiments, the disable program 664 may cause the gate controller 446A to prevent the master module 404A from receiving specimens and/or specimen containers 110 during the shutdown processes and/or while the master module 404A is disabled.

The disable program 664 may also cause the hardware 648 to shut down. For example, fans, lights, and other components may shut down. As described above, while the master module 404A is disabled, the processor 654 or other component may be at least partially active (e.g., a sleep state) and waiting for second instructions generated by the master module controller to enable the master module 404A. In some embodiments, the master module controller 460 may be implemented in the memory 656 as software and may be executed by the processor 654.

The submodules 406A, 406B may be similar to the master module 404A. The submodules 406A, 406B include the submodule controller 462A and the submodule controller 462B, respectively. The submodule controller 462A and the submodule controller 462B perform actions as described with the master module controller 460 to enable and/or disable the submodules 406A, 406B in response to receiving second instructions from the master module 404A.

Referring again to FIGS. 1 and 5, the middleware 138, 238 may generate first instructions to disable specific modules within specific instruments. In other embodiments, the middleware 138, 238 may generate first instructions to disable specific ones of the individual modules 204. The middleware 138, 238 and/or the computer 202 then transmits the first instructions to the instruments or the modules to be disabled. In some embodiments, the middleware 238 may also generate first instructions to cause specific modules to become enabled. The same may apply to the modules 104.

When a module is disabled, the LIS 126 and/or the middleware 138, 238 may make provisions to move testing and/or processing to other modules if needed. For example, if the first submodule 406A (FIG. 4) is disabled, the LIS 126 and/or the middleware 138, 238 may generate instructions to divert specimen containers 110 to the second submodule 406B and/or another module in another instrument. For example, first instructions may be sent to the second instrument 258 and/or the third instrument 260 to run additional tests offloaded from the first submodule 406A. In a similar manner, if one of the individual modules 204 are disabled, the LIS 126 and/or the middleware 138, 238 may reroute the specimen containers destined for the disabled module to a module in one or more of the instruments 254. The same may apply to the modules 104.

The middleware 138, 238 may generate the above-described disable instructions via the first instructions for many reasons. In some embodiments, one or more of the modules (e.g., the modules 404 in the instrument 454) may preform quality control (QC) tests, as described above, that test the integrity of the modules. Quality control test results may be transferred from one or more of the modules and to a respective master module. The QC tests may then be transmitted to the middleware 138, 238 and/or the LIS 126 where one or more programs running therein may analyze the quality control test results and determine if one or more of the modules should be disabled. In the event that the one or more programs determine that one or more modules should be disabled, the middleware 138, 238 may generate first instructions to disable the one or more modules. These first instructions may be transmitted to individual modules 204 or to a master module in one or more of the instruments 254 to disable the specific modules as described above. A user of the diagnostic laboratory system 200 may also enter commands or the like into a local computer that causes the middleware 138, 238 to generate the first instructions to enable and/or disable specific modules.

In some embodiments, the middleware 138, 238 may generate first instructions to disable one or more of the modules in response to a predetermined number of results of the quality control test or tests being outside a predetermined threshold or one or more predetermined thresholds. For example, if a predetermined number of results of the quality control test show a failure in a module, the middleware 138, 238 may generate first instructions to disable the module. In these embodiments, more than one quality control test failure may result in the middleware 138, 238 generating first instructions to disable a module. It is noted that some quality control test results may erroneously show failures. This process of not disabling a module based on a single quality control test result indicating a failure provides that a single erroneous quality control test result will not cause the module associated with the erroneous quality control test result to be disabled.

In some embodiments, the middleware 138, 238 may generate first instructions to disable a module in response to the module having a predetermined number of quality control test results indicating failures within a predetermined time period. In these situations, the module is likely on the brink of failure. Therefore, the middleware 138, 238 may generate the first instructions to disable the module prior to a complete failure of the module.

In some embodiments, the middleware 138, 238 may generate first instructions to disable a module if a predetermined number of consecutive results of a quality control test results indicate failures. Consecutive quality control test failures indicate that a problem exists with the module. Accordingly, the middleware 138, 238 may generate first instructions to disable the module because all tests performed on the specimens will likely be questionable or not usable.

The middleware 138, 238 may generate first instructions to disable one or more of the modules based on other criteria. For example, one or more of the modules may perform hardware tests or other tests. These tests may determine if other parameters of the modules are operating correctly. In response to failures of these tests, the middleware 138, 238 may generate first instructions that disable modules that failed the tests. In other embodiments, one or more of the modules may require products, such as reagents and the like, to process specimens and/or specimen containers. These modules may store an inventory of the required products. The middleware 138, 238 and/or the LIS 126 may monitor inventory of one or more of the modules and the middleware 138, 238 may generate first instructions to disable a module when the inventory of the module is depleted. In other embodiments, one or more of the modules may require scheduled, regular, and/or irregular maintenance. In such embodiments, the middleware 138, 238 may generate first instructions to disable a module when maintenance is due.

In other embodiments, the middleware 138, 238 may generate first instructions to disable one or more modules in response to schedules. In these embodiments, the middleware 138, 238 may generate first instructions to change the operational state of one or more of the modules depending on a time of day. In some embodiments, the LIS 126 may generate a workload for the diagnostic laboratory system 100, 200. When the workload on a first module is light for a period of time, the workload may be shifted to a second module for the period of time and the middleware 138, 238 may generate first instructions to disable the first module for the period of time. In other embodiments, the LIS 126 may generate a workload wherein the first module will not perform any tests for a period of time. Accordingly, the middleware 138, 238 may generate first instructions to disable the first module for the period of time. Disabling the first module reduces wear and tear on the first module and reduces energy consumption of the diagnostic laboratory system 100, 200.

In some embodiments, the diagnostic laboratory system 100, 200 may have 20 or more, 30 or more, 40 or more, 50 or more, 60 or more, 70 or more, 80 or more, 90 or more, or even 100 or more modules 104, 204 and/or instruments 254. The laboratory containing the modules 104, 204 and/or instruments 254 may be very large. For examples, some laboratories may be 100 m by 100 m. Fewer technicians are needed to operate automated laboratories, such as the diagnostic laboratory system 100, 200, so there are few personnel that can manually disable and/or enable individual modules. And, these few personnel may have to travel long distances to reach the modules that would otherwise be manually disabled and/or enabled, which is time consuming. These problems are alleviated by the middleware 138, 238 generating first instructions to disable and/or enable specific modules. Thus, disabling and/or enabling processes can be automated by the first instructions generated by the middleware 138, 238.

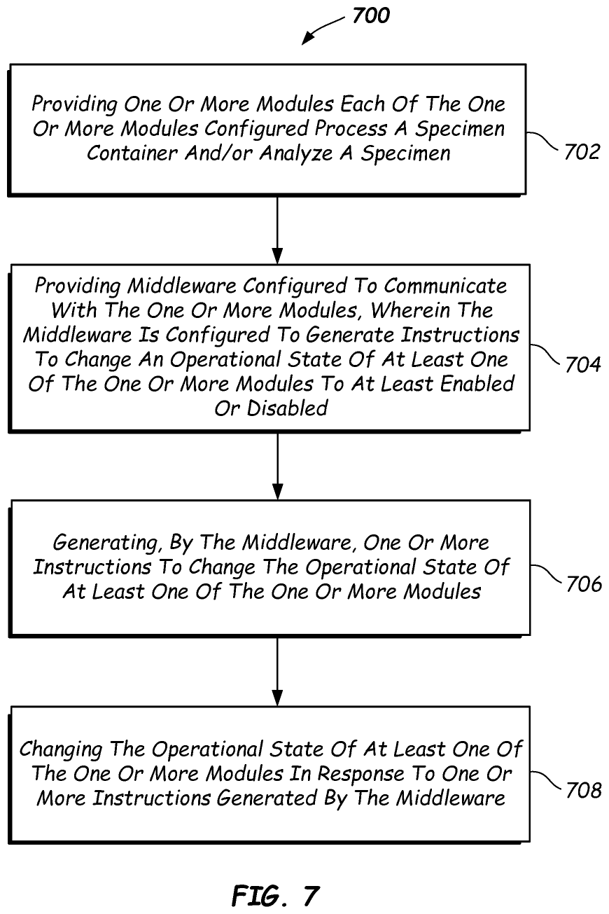

Reference is now made to FIG. 7, which is a flowchart illustrating a method 700 of controlling a diagnostic laboratory system (e.g., diagnostic laboratory system 100, 200). The method 700 includes, in 702, providing one or more modules (e.g., modules 104, 204), each of the one or more modules configured process a specimen container (e.g., specimen container 310) and/or analyze a specimen (e.g., specimen 310A). The method 700 includes, in 704, providing middleware (e.g., middleware 138, 238) configured to communicate with the one or more modules, wherein the middleware is configured to generate instructions to change an operational state of at least one of the one or more modules to at least enabled or disabled. The method 700 includes, in 706, generating, by the middleware, one or more instructions to change the operational state of at least one of the one or more modules. The method 700 includes, in 708, changing the operational state of at least one of the one or more modules in response to one or more instructions generated by the middleware.

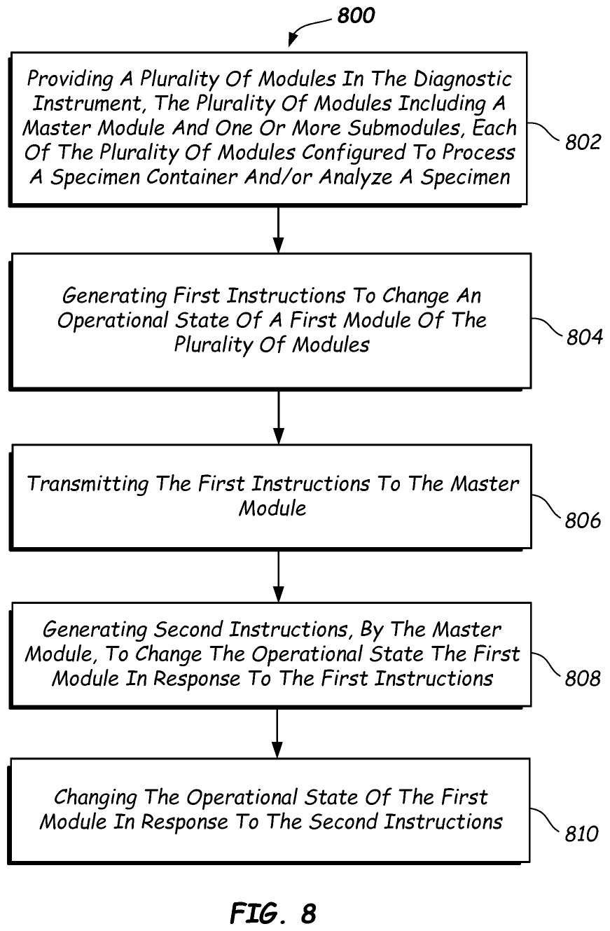

Reference is now made to FIG. 8, which is a flowchart illustrating a method (800) of controlling a diagnostic instrument (e.g., diagnostic instruments 254). The method 800 includes, in 802, providing a plurality of modules (e.g., modules 404) in the diagnostic instrument, the plurality of modules including a master module (e.g., master module 404A) and one or more submodules (e.g., submodules 406), each of the plurality of modules configured to process a specimen container (e.g., specimen container 310) and/or analyze a specimen (e.g., specimen 310A). The method 800 includes, in 804, generating first instructions to change an operational state of a first module of the plurality of modules. The method 800 includes, in 806, transmitting the first instructions to the master module. The method 800 includes, in 808, generating second instructions, by the master module, to change the operational state the first module in response to the first instructions. The method 800 includes, in 810, changing the operational state of the first module in response to the second instructions.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of controlling a diagnostic laboratory system, comprising:
providing one or more modules, each of the one or more modules configured process a specimen container and/or analyze a specimen;
providing middleware configured to communicate with the one or more modules, wherein the middleware is configured to generate instructions to change an operational state of at least one of the one or more modules to at least enabled or disabled;
generating, by the middleware, one or more instructions to change the operational state of at least one of the one or more modules; and
changing the operational state of at least one of the one or more modules in response to one or more instructions generated by the middleware.

2. The method of claim 1, wherein providing one or modules comprises providing one or more modules configured to process a specimen container and one or more modules configured to analyze a specimen.

3. The method of claim 1, wherein:
the middleware is configured to generate instructions to change the operational state of at least one of the one or more modules to enabled, disabled, and sleep;
generating, by the middleware, one or more instructions comprises generating, by the middleware, one or more instructions to change the operational state of at least one of the one or more modules to enabled, disabled, or sleep; and
changing the operational state of at least one of the one or more modules comprises changing the operational state of at least one of the one or more modules to enabled, disabled, or sleep in response to one or more instructions generated by the middleware.

4. The method of claim 1, comprising receiving a status of a first module of the one or modules and wherein generating, by the middleware, one or more instructions comprises generating, by the middleware, one or more instructions to change the operational state of the first module in response to the status of the first module.

5. The method of claim 4, wherein receiving a status of the first module comprises receiving a status indicating that at least one parameter of the first module is outside a predetermined threshold and wherein generating, by the middleware, one or more instructions comprises generating, by the middleware, one or more instructions to change the operational state of the first module to disabled.

6. The method of claim 1, wherein generating, by the middleware, one or more instructions comprises generating, by the middleware, one or more instructions to change an operational state of at least one of the one or more modules depending on a time of day.

7. The method of claim 1, wherein one of the one or more modules is a laboratory analyzer and further comprising running a quality control test on the laboratory analyzer, wherein generating, by the middleware, one or more instructions comprises generating, by the middleware, one or more instructions to change the operational state of the laboratory analyzer in response to at least one result of the quality control test.

8. The method of claim 7, wherein generating, by the middleware, one or more instructions comprises generating, by the middleware, one or more instructions to change the operational state of the laboratory analyzer to disabled in response to a predetermined number of results of the quality control test from the laboratory analyzer being outside of a predetermined threshold.

9. The method of claim 7, wherein generating, by the middleware, one or more instructions comprises generating, by the middleware, one or more instructions to change the operational state of the laboratory analyzer to disabled in response to a predetermined number of results of the quality control test of the laboratory analyzer being outside of a predetermined threshold during a predetermined time period.

10. The method of claim 7, wherein generating, by the middleware, one or more instructions comprises generating, by the middleware, one or more instructions to change the operational state of the laboratory analyzer to disabled in response to a plurality of consecutive results of the quality control test of the laboratory analyzer being outside a predetermined threshold.

11. The method of claim 1, comprising:
generating, by the middleware, one or more instructions to change the operational state a first module of the one or more modules to disabled; and
routing specimen containers to bypass the first module in response to the operational state of the first module being disabled.

12. The method of claim 1, comprising:
generating, by the middleware, one or more instructions to change the operational state a first module of the one or more modules to enabled; and
routing specimen containers to the first module in response to the operational state of the first module being enabled.

13. The method of claim 1, comprising:
generating, by the middleware, one or more instructions to change the operational state a first module of the one or more modules to disabled, wherein the first module is configured to perform at least a first process on specimen containers or specimens when the operational state of the first module is enabled;
generating, by the middleware, one or more instructions to change the operational state a second module of the one or more modules to enabled, wherein the second module is configured to perform at least the first process on specimen containers or specimens when the operational state of the second module is enabled; and
performing the first process on specimen containers and/or specimens using the second module.

14. The method of claim 1, comprising:
providing a laboratory information system in communication with the middleware, wherein the laboratory information system is configured to schedule processing on the one or more modules,
wherein generating, by the middleware, one or more instructions comprises generating, by the middleware, one or more instructions to change an operational state of at least one of the one or more modules in response to scheduling by the laboratory information system.

15. The method of claim 1, wherein
providing one or more modules comprises providing a master module and one or more submodules in communication with the master module;
providing middleware comprises providing middleware configured to communicate with the master module;
generating, by the middleware, one or more first instructions to change an operational state of at least one of the master module or the one or more submodules;
transmitting the first instructions to the master module;
generating, by the master module, second instructions to change the operational state of at least one of the master module or the one or more submodules; and
changing the operational state of at least one of the master module or the one or more submodules in response the second instructions.

16. A method of controlling a diagnostic instrument, comprising:
providing a plurality of modules in the diagnostic instrument, the plurality of modules including a master module and one or more submodules, each of the plurality of modules configured to process a specimen container and/or analyze a specimen;
generating first instructions to change an operational state of a first module of the plurality of modules;
transmitting the first instructions to the master module;
generating second instructions, by the master module, to change the operational state the first module in response to the first instructions; and
changing the operational state of the first module in response to the second instructions.

17. The method of claim 16, comprising providing a computer configured to communicate with the master module, wherein generating the first instructions comprises generating the first instructions using the computer.

18. The method of claim 17, comprising running middleware on the computer, wherein generating the first instructions comprises generating the first instructions using the middleware.

19. The method of claim 16, wherein generating the first instructions comprises generating first instructions to change the operational state of the first module to enabled or disabled.

20. The method of claim 16, generating the first instructions comprises generating first instructions to change the operational state of the first module to enabled, disabled, or sleep.

21. The method of claim 16, comprising determining a status of the first module and wherein generating the first instructions comprises generating the first instructions to change the operational state of the first module in response to the status of the first module.

22. The method of claim 21, wherein receiving a status of the first module comprises receiving a status indicating that at least one parameter of the first module is outside a predetermined threshold and wherein generating the first instructions comprises generating first instructions to change the operational state of the first module to disabled.

23. The method of claim 16, wherein generating first instructions comprises generating first instructions to change the operational state of the first module depending on a time of day.

24. The method of claim 16, wherein the first module is a laboratory analyzer and further comprising running a quality control test on the first module, wherein generating the first instructions comprises generating the first instructions to change the operational state of the first module in response to at least one result of the quality control test.

25. The method of claim 24, wherein generating first instructions to change the operational state of the first module comprises generating first instructions to change the operational state of the first module to disabled in response to a predetermined number of results of the quality control test from the first module being outside of a predetermined threshold.

26. The method of claim 24, wherein generating first instructions to change the operational state of the first module comprises generating first instructions to change the operational state of the first module to disabled in response to a predetermined number of results of the quality control test of the first module being outside of a predetermined threshold during a predetermined time period.

27. The method of claim 24, wherein generating first instructions to change the operational state of the first module comprises generating first instructions to change the operational state of the first module to disabled in response to a plurality of consecutive results of the quality control test of the first module being outside a predetermined threshold.

28. The method of claim 16, comprising:
generating first instructions to change the operational state of the first module to disabled; and
routing specimen containers to bypass the first module in response to the operational state of the first module being disabled.

29. The method of claim 16, comprising:
generating first instructions to change the operational state of the first module to enabled; and
routing specimen containers to the first module in response to the operational state of the first module being enabled.

30. A diagnostic instrument, comprising:
a master module; and
one or more submodules in communication with the master module,
wherein the master module is configured to receive first instructions to change an operational status of at least one of the one or more submodules and to generate second instructions to change the operational status of at least one of the one or more submodules in response to the first instructions, and
wherein the one or more submodules are configured to change operational state in response to the second instructions.
